(19) United States
(12) Patent Application Publication
(10) Pub. No.: US 2009/0062806 A1
(43) Pub. Date: Mar. 5, 2009

(54) Tibial preparation apparatus and method

(76) Inventors: James W. Scott, Tifton, GA (US); Robert G. Metzger, Wakarusa, IN (US)

Correspondence Address: HARNESS, DICKEY & PIERCE, P.L.C., P.O. BOX 828, BLOOMFIELD HILLS, MI, 48303, US

(21) Appl. No.: 10/702,335

(22) Filed: Nov. 6, 2003

(51) Int. Cl. A61B 17/17

(52) U.S. Cl. 606/84; 606/86 R

(57) ABSTRACT

An apparatus and method for preparing a tibia for knee surgery. The apparatus includes a tibial base having a center axis, and a handle coupled to the base at an anterior base location that is offset relative to the center axis.

### FIELD OF THE INVENTION

The present invention relates to an apparatus and method for tibial preparation.

### BACKGROUND OF THE INVENTION

During knee replacement surgery the tibial surface may be prepared for implantation of a tibial component of a knee implant. Several preparation instruments may be used to assist the surgeon in this procedure. A surgeon generally uses, for example, a tibial template and/or trial tibial tray to determine the tibial implant size, as well as various cutting, punching or reaming guides to make the appropriate cuts or reamed surfaces in the bone, and to ensure a proper alignment prior to implanting the tibial component.

The tibial preparation procedure typically starts with making an initial tibial plateau cut on the proximal tibia of the knee joint. Using a tibial template, the size of the trial tray is then selected and placed over the resected surface of the tibia. After trial reduction, the trial tray is removed and a punch guide is placed on the resected tibial surface. The tibial bone is cut or reamed through openings in the punch guide and trial tray to prepare the bone to receive the tibial component of the implant.

During the tibial preparation procedure, the surgeon may need to remove and reposition or re-orient a preparation instrument using a handle attached to the instrument. In the existing art, the handle is centrally attached to the instrument, and is often removed and re-attached, to avoid tissue impingement or provide clearance, for example. The prior art handle may obscure the view of the trial trays or guides and make alignment evaluation cumbersome, and generally reduces the manipulative clearance available to the surgeon.

Accordingly, improved tibial preparation instruments that avoid some of the problems associated with the prior art handles are desirable.

### SUMMARY OF THE INVENTION

One embodiment of the invention provides an apparatus for preparing a tibia for knee surgery. The apparatus includes a tibial base having a center axis, and a handle coupled to the base at an anterior base location that is offset relative to the center axis.

Another embodiment of the invention provides an apparatus for preparing a tibia for knee surgery. The apparatus includes a reversible base that is operable to engage in contact a surface of the tibia and has a center axis. The apparatus also includes a link coupled to the base at a position that is medially offset from the center axis. A handle is coupled to the link.

Another embodiment of the invention provides an apparatus for preparing a tibia in knee surgery. The apparatus includes a tibial base having a center axis, and a handle coupled to the base at a first end. The first end has a medially offset cutout oriented to provided a lateral clearance relative to the center axis of the base.

Another embodiment of the invention provides a method for tibial preparation for a knee implant procedure. The method includes selecting a tibial base, and coupling the tibial base with a handle at a position medially offset relate to a center axis of the tibial base.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of an embodiment of a tibial preparation apparatus according to the present invention, the apparatus shown in the environment of a right knee tibial bone.

### DETAILED DESCRIPTION OF THE PREFERRED EMBODIMENTS

The following description of the embodiments is merely exemplary in nature and is in no way intended to limit the invention, its application, or uses.

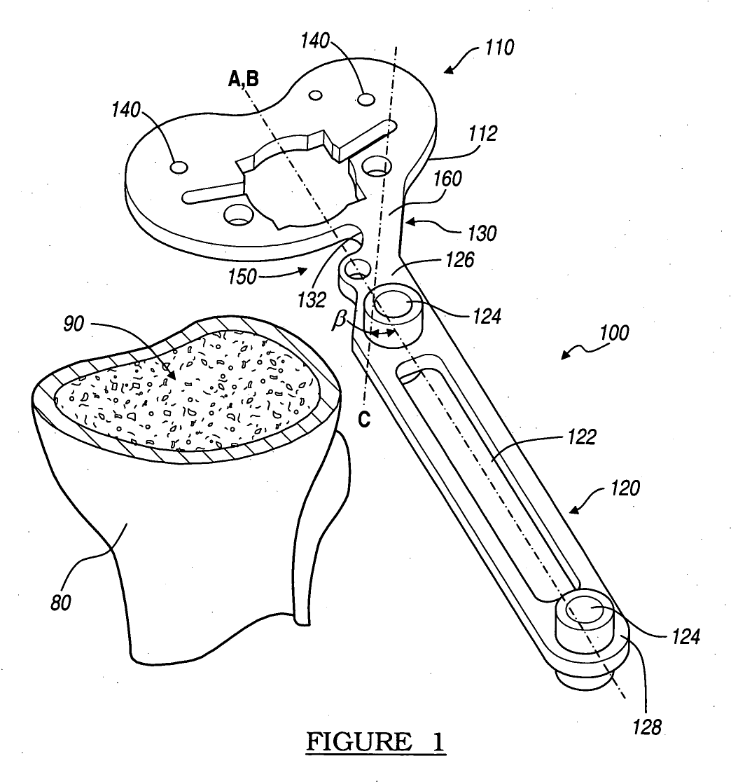

FIG. 1 illustrates an embodiment for an apparatus 100 used to prepare a tibial surface 90 for knee surgery. The apparatus 100 is shown in the exemplary environment of a right knee tibial bone 80. The apparatus 100 includes a tibial base 110, a handle 120, and a link 130. The tibial base 110 can be a tibial template, a trial tibial tray or plate, a punching, reaming, cutting, or alignment guide or jig, or any other component that may be placed on the tibial surface 90 in preparation for knee replacement surgery. The tibial base 110 may be reversible in some embodiments, such as, for example when the tibial base 110 is a tibial template. The tibial base has a periphery 112 and a center axis “A”, which is generally, but not necessarily, an axis of symmetry of the tibial base 110. The tibial base 110 may be temporarily secured to the tibial surface 90 with pins (not shown) inserted through fixation holes 140. The center axis A may lie on the median plane of the tibia 80 when the tibial base 110 is positioned on the tibial surface 90.

The handle 120 is an elongated member that may include a longitudinal slot 122 to provide weight reduction to the apparatus 100 without compromising its strength. The handle 120 may include one or more sleeves 124 for inserting alignment rods, such as rods or pins or drill bits for setting rotation along the tibial tubercle or second metatarsal, for example. The handle 120 has a proximal end 126, a distal end 128, and a longitudinal axis “B” which coincides with the center axis A of the tibial base 110 when the handle 120 is attached to the tibial base 110. The handle 120 is used to conveniently move the base 110 relatively to the tibial surface 90, but it can also be used to verify rotational, varus/valgus, and flexion/extension alignments, for example.

Although the handle 120 may be centered about the center axis A of the tibial base 110, such that the longitudinal axis B of the handle coincides with the center axis A of the base 110, at least a partial offset in the medial direction (“medial offset”) is provided at the base-handle connection. The medial offset, generally designated as 150, may be constructed by a variety of structures in combination with a handle 120 that is integral to the base 110, or a modular handle 120 that can be removably attached to the base 110, or with a link 130 used as an intermediate structure between the base 110 and the handle 120.

In one embodiment, the link 130 extends from a medially offset anterior position 160 on the periphery 112 of the base 110, and couples the base 110 to the proximal end 126 of the handle 120. The link 130 has a center axis C which is medially offset from the axis A at an angle β, which provides a measure for the medial offset 150. The angle β may be, for example, about 15° to about 45° degrees.

The medial offset 150 provides a desirable clearance that helps avoid lateral tissue impingement during the tibial preparation procedures, which include trial sizing, alignment, punching, reaming, cutting, etc. Furthermore, the medial offset 150 enhances the visibility of the tibial base 110, the periphery 112 of the tibial base 110 and the tibial bone 80, and also provides manipulative clearance for the surgeon.

The link 130 may include a curved portion 132 which provides a smooth transition between the base 110 and the handle 120. The link 130 may be modular, or integral to the base 110, or integral to the handle 120. Accordingly, the link 130 can be removably coupled to the base 110 or to the handle 120 or to both. In another embodiment, the handle 120 is directly coupled to the tibial base 110 at the medially offset anterior position 160 and includes a cutout that defines the medial offset 150.

In operation, the apparatus 100 is selectively placed on the tibial surface 90, such that the medially offset position 160 is on the medial and anterior side of each knee. For a reversible base 110, this is accomplished by rotating the apparatus 100 by 180° for use from a right knee to a left knee, and conversely. In applications in which the tibial base 100 is not reversible, two mirror image apparatuses 100, one for each knee, can be provided.

The description of the invention is merely exemplary in nature and, thus, variations that do not depart from the gist of the invention are intended to be within the scope of the invention. Such variations are not to be regarded as a departure from the spirit and scope of the invention.

1. An apparatus for preparing a tibia for knee surgery, the apparatus comprising:
a tibial base having a center axis; and
a handle having a longitudinal axis coaxially aligned to the center axis of the tibial base, the handle coupled to the tibial base only at one location on a periphery of the tibial base, the location being offset relative to the center axis and defining a clearance for avoiding tissue impingement.

2. The apparatus of claim 1, wherein the base is selected from the group consisting of a tibial template, a trial tray, a punch guide, a cutting guide, a reaming guide, an alignment guide, and combinations thereof.

3. (canceled)

4. The apparatus of claim 1, further including a link extending from the base at an offset relative to the center axis and coupled with the handle.

5. The apparatus of claim 4, wherein the offset is in a medial direction when the base engages a surface of the tibia.

6. The apparatus of claim 1, wherein the base is reversible such that the offset is in the medial direction for right and left knee surgery.

7. (canceled)

8. An apparatus for preparing a tibia for knee surgery, the apparatus comprising:
a reversible base operable to engage and contact a surface of the tibia, the base having a center axis;
only one link having first and second ends, the first end coupled to the base at only one position that is medially or laterally offset from the center axis and defining a clearance for avoiding tissue impingement; and
a handle having a central longitudinal axis coaxially aligned with the center axis of the base, the handle coupled to the second end of the link.

9-10. (canceled)

11. The apparatus of claim 8, wherein the link and the handle are integral to the base.

12. The apparatus of claim 8, wherein the link is integral to the handle.

13. (canceled)

14. The apparatus of claim 8, wherein the link defines a longitudinal axis that is at an angle with the center axis of the base.

15. The apparatus of claim 14, wherein the angle is about 15° to about 45°.

16. The apparatus of claim 8, wherein the link has a curved portion between the base and the handle, the curved portion defining the clearance, the clearance extending beyond the center axis of the base.

17. An apparatus for preparing a tibia in knee surgery, the apparatus comprising:
a tibial base having a center axis; and
a handle having a central longitudinal axis substantially parallel to the center axis of the tibial base, the handle having a first end, the first end coupled to the base only at one location offset relative to the center axis, the first end having a medially offset cutout, the cutout defining a lateral clearance for avoiding tissue impingement, the lateral clearance extending beyond the center axis of the base and beyond the longitudinal axis of the handle.

18. (canceled)

19. The apparatus of claim 17, wherein the cutout has a curved portion adjacent to the base.

20-26. (canceled)

27. The apparatus of claim 1, wherein the center axis lies on a median plane of the tibia when the tibial base is positioned on a tibial surface.

28. The apparatus of claim 1, wherein the center axis is coplanar with the tibial base.

29. The apparatus of claim 8, wherein the center axis is coplanar with the base.

30. The apparatus of claim 8, wherein the center axis lies on a median plane of the tibia when the base is positioned on the surface of the tibia.

31. An apparatus for preparing a tibia in knee surgery, the apparatus comprising:
a tibial base; and
a handle extending substantially along a longitudinal axis, the handle having first and second ends, the first end of the handle attached to the tibial base at only one location of the tibial base that is offset from the longitudinal axis of the handle for avoiding tissue impingement.

32. The apparatus of claim 31, wherein the first end includes a cutout defining a clearance between the longitudinal axis and the location of attachment to the tibial base.
